(19) United States
(12) Patent Application Publication
(10) Pub. No.: US 2025/0135185 A1
(43) Pub. Date: May 1, 2025

(54) PUMP SYSTEMS AND METHODS

(71) Applicant: Shifamed Holdings, LLC, Campbell, CA (US)

(72) Inventors: Jonelle BALDUCCI, Campbell, CA (US); Ari RYAN, Sunnyvale, CA (US)

(21) Appl. No.: 18/835,590

(22) PCT Filed: Feb. 2, 2023
(86) PCT No.: PCT/US2023/061874

§ 371 Date: Aug. 2, 2024

Related U.S. Application Data
(60) Provisional application No. 63/267,476, filed on Feb. 2, 2022.

(51) Int. Cl. A61M 60/90; A61M 60/122; A61M 60/216; A61M 60/81; A61M 60/865

(52) U.S. Cl. CPC A61M 60/90; A61M 60/122; A61M 60/216; A61M 60/81; A61M 60/865; A61M 2205/04; A61M 2205/103

(57) ABSTRACT

Catheter blood pumps that include an expandable conduit coupled to at least one hub shaped to promote smooth blood flow. In some examples, the at least one hub includes non-metallic configuration and the struts comprise a metallic configuration (e.g., nitinol). The hub can include one or more layers, including a combination of Chronoflex and Pebax.

### CROSS REFERENCE TO RELATED APPLICATIONS

This application claims the benefit of priority to U.S. Application No. 63/267,476, filed Feb. 2, 2022, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. Intra-aortic balloon pumps (IABP) are used to support circulatory function, such as treating heart failure patients. An IABP is typically placed within the aorta, and inflated and deflated in counter-pulsation fashion with the heart contractions, with one function being to provide additive support to the circulatory system. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

More recently, minimally invasive rotary blood pumps have been developed, which are inserted into the body in connection with the cardiovascular system to pump arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient. The smallest rotary blood pumps currently available may be percutaneously inserted into the vasculature of a patient through an access sheath, thereby avoiding more extensive surgical intervention, or through a vascular access graft. One such device is a percutaneously inserted ventricular support device.

Although blood pumps exist, there is a need to provide additional improvements in the field of ventricular support devices and similar blood pumps for treating compromised cardiac blood flow.

### BRIEF DESCRIPTION OF THE DRAWINGS

FIG. 1 is a side view of an exemplary blood pump that includes an expandable scaffold that supports a blood conduit with an impeller housed therein.

FIGS. 2A-2B illustrate one embodiment of a hub assembly and distal tip of a blood pump.

FIG. 3 is a flowchart indicating an exemplary method of using a blood pump.

### SUMMARY

A distal tip of an intravascular blood pump is provided, comprising an elongate portion configured to be coupled to the intravascular blood pump; a curved distal portion distal to the elongate portion; a self-sealing septum disposed at a distal end of the curved distal portion; and a lumen passing through the elongate portion and the curved distal portion and terminating prior to the self-sealing septum, wherein the self-sealing septum is configured to be punctured by a guidewire to allow the guidewire to gain access to the lumen, wherein the self-sealing septum is configured to self-seal upon removal of the guidewire to prevent blood or fluids from entering the lumen.

In one aspect, the curved distal portion comprises a J-tip.

In one aspect, the curved distal portion comprises a pigtail.

In one aspect, the distal tip further comprises a guide feature configured to direct a guidewire through the self-sealing septum to the lumen.

An intravascular blood pump is provided, comprising: a collapsible conduit having an inner lumen for passing fluid therethrough, the conduit comprising a proximal end having a proximal opening, and a distal end having a distal opening; at least one impeller within the conduit, the at least one impeller arranged to pump fluid into the distal opening of the conduit and out of the proximal opening of the conduit; a plurality of struts extending from the proximal end or the distal end of the conduit; and a hub configured to receive the plurality of struts; and a distal tip connected to the hub, the distal tip comprising a proximal section, a central section, and a distal section, wherein the central section is stiffer than the proximal section and the distal section.

In one aspect, the central section has a wall thickness that is greater than a wall thickness of the proximal section and the distal section.

In one aspect, the distal section is attached to the proximal section at the central section.

In one aspect, the proximal section includes a male shaped fitting configured to mate with a female shaped fitting of the distal section.

In one aspect, the pump further comprises an atraumatic tip coupled to the distal section.

In one aspect, the pump further comprises, a guidewire lumen disposed within the distal tip.

In one aspect, the pump further comprises a self-sealing septum disposed at a distal end of the guidewire lumen.

In one aspect, the self-sealing septum is configured to be punctured to allow a guidewire to pass through.

In one aspect, The pump of claim 12, wherein the self-sealing septum is configured to re-seal itself when the guidewire is removed.

A method of delivering an intravascular blood pump, the method comprising: piercing a self-sealing septum of a distal tip with a guidewire to insert the guidewire into the blood pump; positioning the blood pump at a target tissue location with the guidewire; and retracting the guidewire into the distal tip to cause the self-sealing septum to re-seal.

### DETAILED DESCRIPTION

The present disclosure is related to medical devices, systems, and methods of use and manufacture. In particular, described herein are pumps adapted to be disposed within a physiologic vessel, wherein the distal pump portion includes one or more components that act upon fluid. For example, the pumps herein may include one or more rotating members that when rotated, can facilitate the movement of a fluid such as blood.

Any of the disclosure herein relating to an aspect of a system, device, or method of use can be incorporated with any other suitable disclosure herein. For example, a figure describing only one aspect of a device or method can be included with other embodiments even if that is not specifically stated in a description of one or both parts of the disclosure. It is thus understood that combinations of different portions of this disclosure are included herein.

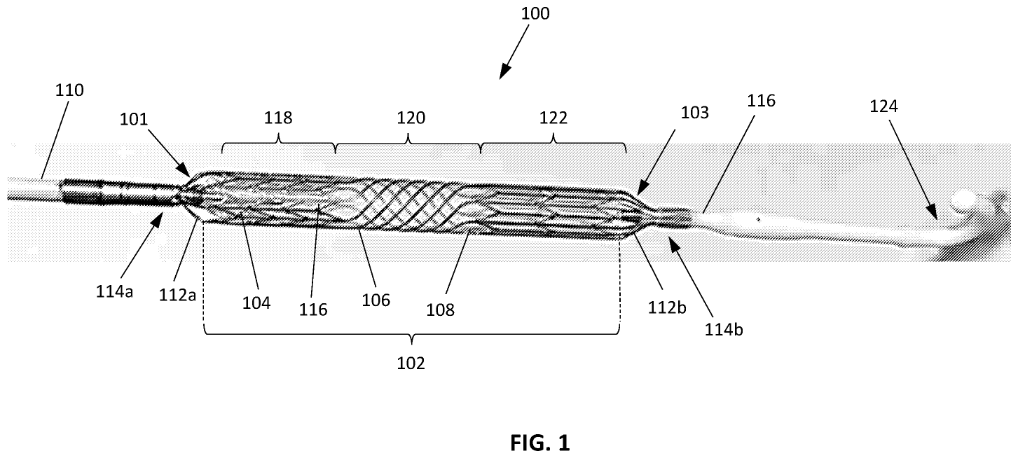

FIG. 1 shows a side view of one embodiment of an intravascular catheter blood pump 100. The blood pump 100 includes an expandable/collapsible blood conduit 102 that is configured to transition between an expanded state, as shown in FIG. 1, and a collapsed state (not shown). For example, the conduit 102 may be in the collapsed state when confined within a delivery catheter for delivery to the heart, expanded upon release from the delivery catheter for blood pumping, and collapsed back down within the delivery catheter (or other catheter) for removal from heart. When in the expanded state, the conduit 102 is radially expanded so as to form an inner lumen for passing blood therethrough. In some embodiments, the conduit 102 is impermeable to blood. When in the expanded state, the inner lumen of the conduit 102 may be configured to accommodate blood pumped by one or more impellers therein. The one or more impellers may be collapsible so that they may collapse to a smaller diameter when the conduit 102 is in the collapsed state. The one or more impellers may be positioned within one or more impeller regions of the conduit 102. In some examples, the impeller region(s) of the conduit 102 is/are radially stiffer than other regions (e.g., adjacent regions) of the conduit 102 to prevent the impeller(s) from contacting the interior walls of the conduit 102.

In this example, the blood pump 100 includes an impeller 104 within a proximal portion of the conduit 102. In some cases, the blood pump 100 can include more than one impeller. For example, the blood pump 100 may include a second impeller in a distal region 122 of the fluid conduit 102. In some cases, blood pump 100 may include more than two impellers. The conduit 102 includes a first (e.g., proximal) end having a first (e.g., proximal) opening 101, and a second (e.g., distal) end having a second (e.g., distal) opening 103. The first opening 101 and second opening 103 may be configured as and an inlet and outlet for blood. For example, blood may largely enter the conduit 102 via the second (e.g., distal) opening 103 and exit the conduit 102 via the first (e.g., proximal) opening 101. In such case, the second opening 103 acts as a blood inlet and the first opening 101 acts as a blood outlet. The one or more impellers (e.g., impeller 104) may be configured to pump blood from the inlet toward the outlet. In an exemplary operating position, the second opening 103 (e.g., inlet) may be distal to the aortic valve, in the left ventricle, and the first opening 101 (e.g., outlet) may be proximal to the aortic valve (e.g., in the ascending aorta).

The conduit 102 can include a tubular expandable/collapsible scaffold 106 that provides structural support for a membrane 108 that covers at least a portion of inner surfaces and/or outer surfaces of the scaffold 106. The scaffold 106 includes a material having a pattern of openings with the membrane 108 covering the openings to retain the blood within the lumen of the conduit 102. The scaffold 106 may be unitary and may be made of a single piece of material. For example, the scaffold 106 may be formed by cutting (e.g., laser cutting) a tubular shaped material. Exemplary materials for the scaffold 106 may include one or more of: nitinol, cobalt alloys, and polymers, although other materials may be used.

The blood pump 100 can further include proximal struts 112a that extend from the scaffold 106 near the first opening 101 (e.g., blood outlet region) and distal struts 112b that extend from the scaffold 106 near the second opening 103 (e.g., blood inlet region). The proximal struts 112a are coupled to first hub 114a of a proximal shaft 110. The distal struts 112b are coupled to second hub 114b of a distal portion 114. In this example, the first hub 114a includes a bearing assembly through which a central drive cable 116 extends. The drive cable 116 is operationally coupled to and configured to rotate the impeller 104.

In some cases, the impeller 104 is fully positioned axially within the conduit 102. In other cases, a proximal portion of the impeller 104 is positioned at least partially outside of the conduit 102. That is, at least a portion of the impeller may be positioned in axially alignment with a distal portion of the struts 112a.

The conduit 102 and the scaffold 106 may characterized as having a proximal region 118, a central region 120, and a distal region 122. The central region 120 may be configured to be placed across a valve (e.g., aortic valve) such that the proximal region 118 is at least partially within a first heart region (e.g., ascending aorta) and the distal region 122 is at least partially within a second heart region (e.g., left ventricle). In some embodiments, the central portion may be more flexible than the proximal and distal regions. The proximal region 118 (and in some cases the distal region 122) may be configured to house an impeller therein. The proximal region 118 may (and in some cases the distal region 122) has a stiffness sufficient to withstand deformation during operation of the blood pump 100 when within the beating heart and to maintain clearance (i.e., a gap) between an impeller region of the blood pump 100 and the rotating impeller 104. The distal region 122 includes the second (e.g., distal) opening 103 of the conduit 102, and may serve as the blood inlet for the conduit 102.

The central region 120 may be less rigid relative to the proximal region 118 (and in some cases the distal region 122). The higher flexibility of the central region 120 may allow the central region 120 to deflect when a lateral force is applied on a side of the conduit 102, for example, as the conduit 102 traverses through the patient's blood vessels and/or within the heart. For example, the central region 120 may be configured to laterally bend upon a lateral force applied to the distal region 122 and/or the proximal region 118. In some cases, it may be desirable for the central region 120 to laterally bend as the conduit 102 traverses the ascending aorta and temporarily assume a bent configuration when the conduit 102 is positioned across an aortic valve. In this example, the central region 120 includes a helical arrangement of longitudinally running elongate elements configured to provide flexibility for lateral bending. In some examples, a distal tip 124 of the blood pump 100 has a curved portion to form an atraumatic tip. In some cases, the distal tip 124 is flexible (e.g., laterally bendable) to enhance the atraumatic aspects of the distal tip 124. For example, the distal tip 124 may be sufficiently flexible to bend when pressed against tissue (e.g., by a predetermined amount of force) to prevent puncture of the tissue.

The first hub 114a (e.g., proximal hub) and/or the second hub 114b (e.g., distal hub) may include features that promote smooth blood flow into and/or out of the conduit 102. The first and second hubs may further include features for attaching or connecting to the struts, scaffold, and/or conduit of the blood pump. Such features may prevent or reduce the occurrence of stagnant and/or turbulent blood flow that may otherwise tend to occur in regions near the first opening 101 (e.g., outlet region) and/or the second opening 103 (e.g., inlet region) of the conduit 102. Since stagnant and/or turbulent blood flow is associated with blood coagulation and/or clotting, measures to reduce this can be beneficial to for patient outcome.

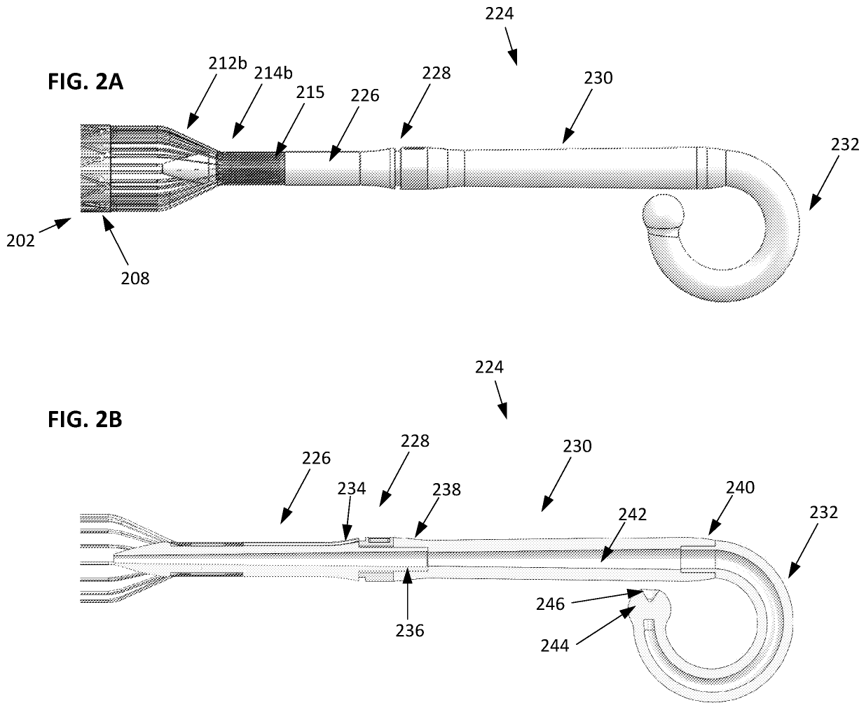

FIG. 2A-2B illustrate components of a blood pump with an exemplary distal hub 214b configured to attach to the struts 212b of the blood conduit and further configured to promote non-turbulent fluid flow past/through the hub and struts and into the conduit. Additionally, the configuration of the distal hub and its attachment to the struts can be designed and configured to prevent, reduce, or limit areas of stagnant blood flow, particularly under the distal struts of the scaffold, to prevent, reduce, or limit clot formation at or near the hub or struts. FIG. 2A shows a side view of a distal end of a conduit 202, which includes a membrane 208 covering a portion of struts (e.g., distal struts) 212b that extend from the distal end of the conduit 202.

FIG. 2A also shows an attachment region 215 that defines the location in the hub 214b where the struts 212b are connected or attached to the hub. Any number of struts 212b (e.g., 1, 2, 3, 4, 5, 6, 7, 8, 9, 10, 11 or more, etc.) can be implemented in the blood pump and attached to the hub. In some exemplary embodiments, the blood pump includes only 4, 5, 6, 7, or 8 struts.

Referring still to FIGS. 2A-2B, the distal tip 224 of the blood pump can include a plurality of sections that enable additional functionality from the distal tip, including sections of variable or selected stiffness. For example, the distal tip 224 can include a proximal section 226 that includes or is attached to the distal hub 214b, a central section 228, a distal section 230, and a curved or atraumatic section 232, as shown. In one example, the central section 228 can be less flexible (or stiffer) than the proximal section 226 and the distal section 230.

In some embodiments, the stiffness of the distal tip 224 can be controlled along its length by creating sections with increased wall thickness relative to the other sections. Referring to the cross-sectional view of FIG. 2B, it can be seen that the sections of the distal tip can comprise separate components which can be press fit or joined together. As shown, proximal section 226 can include a first portion 234 having a first outer diameter and a second portion 236 having an outer diameter less than that of the first outer diameter. Similarly, the distal portion 230 can include a first portion 238 configured to slide over or mount/mate directly with the second portion 236 of the proximal section 226. In the illustrated example, the second portion of the proximal section can include a male fitting, and the first portion of the distal section can include a female fitting configured to slide over the male fitting of the proximal section. The central section of the distal tip results from the joining of these two sections, and has an increased stiffness relative to the proximal section or distal section alone due to larger wall thickness in this area. A similar junction/connection can be seen at section 240 where the atraumatic section 232 is joined to the distal section 230.

The distal tip configuration shown in FIG. 2B therefore allows for varying stiffness/flexibility along the length of the distal tip. These areas of increased stiffness can be chosen and positioned according to design needs or the targeted anatomy. As such, the distal tip can be designed to bend and or deflect in certain sections (e.g., in the blood conduit 202, the proximal section 226, or in the distal section 230) while retaining rigidity or stiffness in other sections (e.g., in the central section 228 or in section 240 immediately proximal to the atraumatic tip.

Still referring to FIG. 2B, the distal tip 224 can further include a lumen 242 adapted to receive additional tools such as a guidewire. In use, the guidewire can be advanced through the blood pump, through the lumen 242, and out through the end of the distal tip. In one embodiment, the distal tip 224 can include a self-sealing septum 244 positioned distally from the distal end of the lumen, as shown. It is noted that in this embodiment, initially, the lumen 242 does not extend fully through the distal tip 224, but instead terminates just short of the self-sealing septum. The self-sealing septum 244 is configured to be punctured by the guidewire prior to delivery of the blood pump. Puncturing the self-sealing septum with the guidewire can create a path between the distal end of the lumen and the distal end of the distal tip. As shown in FIG. 2B, the distal tip can include a guide feature 246 configured to direct the guidewire from the distal end of the distal tip towards the lumen 242 when the guidewire is used to puncture the self-sealing septum. The guide feature 246 can comprise, for example, a conical or tapering shape to promote alignment of the guidewire with the lumen. Once the blood pump is positioned in the treatment location over the guidewire, the guidewire can be removed from the blood pump and distal tip. As the guidewire is removed from the self-sealing septum, the self-sealing septum can be configured to seal off the distal end of lumen 242 within the distal tip 224 to prevent contamination of the device during use. Additionally, the self-sealing septum can prevent entry of blood into the lumen, which can prevent clotting from forming within the distal tip of the device during therapy/use and also prevent clotting from exiting the lumen into the blood pump. In some embodiments, the device and/or distal tip can be primed with heparanized saline to further prevent clotting during use.

In some embodiments, the dimensions, diameter, and material properties of the seal-healing septum 244 can be optimized to promote self-healing when the guidewire is removed. For example, in some embodiments, the self-healing septum can include a diameter ranging between: ˜0.06″-0.14″. In general, smaller diameters do not self-heal as effectively. Larger diameters are easier to puncture and seal but are limited by the overall profile of the device. The septum can further comprise a thickness ranging between: ˜0.050″-0.1″, but thicknesses outside of that range could also work depending on the durometer of the material. Suitable materials include silicone, urethanes, or thermoplastic polyurethanes, for example, having a durometer ranging between: 20A-50A

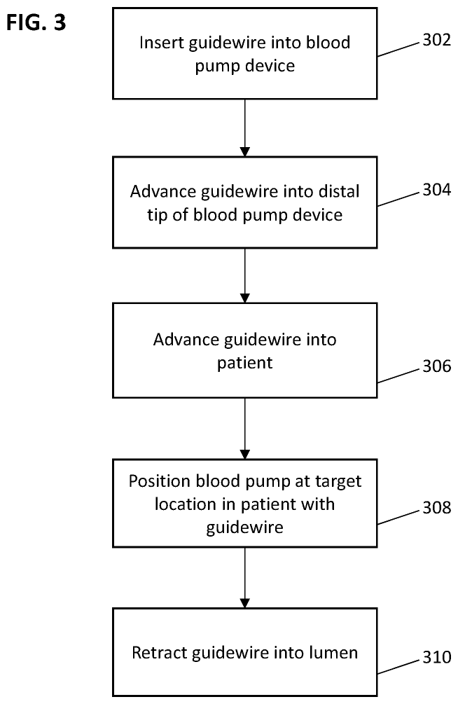

FIG. 3 is a flowchart describing a method of using the blood pump described above having a distal tip and a self-sealing septum. At step 302, a guidewire can be inserted into the blood pump. At step 304, the guidewire is advanced through the lumen of the distal tip. In some embodiments, advancing the guidewire through the distal tip punctures or forms a passage between the distal end of the distal tip and the guidewire lumen through the self-sealing septum. At step 306, the guidewire can be advanced into a body lumen, cavity, or organ of the patient. At step 308, the blood pump can be positioned at a target tissue location with the guidewire. Finally, at step 310, the guidewire can be retracted back into the lumen of the distal tip, and the self-sealing septum can re-seal itself to prevent contamination or bodily fluids from entering the blood pump device distal tip.

Although the above examples show and describe a distal hub that is distal to the blood conduit, in some cases, a proximal hub having similar non-turbulent flow promoting features (e.g., channels) may be positioned proximal to the conduit. For example, a proximal hub may have a body and spokes shaped to form channels that promote non-turbulent flow out of a proximal opening (e.g., outlet) of the conduit. Such proximal hub may be used with or without the non-turbulent flow promoting distal hub shown.

Any of the blood pumps described herein may include surfaces with one or more anticoagulant agents. For example, at least a portion of one or more of the hubs, conduits (e.g., scaffold and/or membrane), struts (e.g., proximal and/or distal struts), distal tips and/or impellers of the blood pumps described herein may include a coating or material having an anticoagulant agent. In some cases, the anticoagulant agents may include drugs such as heparin, warfarin and/or prostaglandins.

What is claimed is:

1. A distal tip of an intravascular blood pump, comprising:
an elongate portion configured to be coupled to the intravascular blood pump;
a curved distal portion distal to the elongate portion;
a self-sealing septum disposed at a distal end of the curved distal portion; and
a lumen passing through the elongate portion and the curved distal portion and terminating prior to the self-sealing septum,
wherein the self-sealing septum is configured to be punctured by a guidewire to allow the guidewire to gain access to the lumen, wherein the self-sealing septum is configured to self-seal upon removal of the guidewire to prevent blood or fluids from entering the lumen.

2. The distal tip of claim 1, wherein the curved distal portion comprises a J-tip.

3. The distal tip of claim 1, wherein the curved distal portion comprises a pigtail.

4. The distal tip of claim 1, further comprising a guide feature configured to direct a guidewire through the self-sealing septum to the lumen.

5. An intravascular blood pump, comprising:
a collapsible conduit having an inner lumen for passing fluid therethrough, the conduit comprising a proximal end having a proximal opening, and a distal end having a distal opening;
at least one impeller within the conduit, the at least one impeller arranged to pump fluid into the distal opening of the conduit and out of the proximal opening of the conduit;
a plurality of struts extending from the proximal end or the distal end of the conduit; and
a hub configured to receive the plurality of struts; and
a distal tip connected to the hub, the distal tip comprising a proximal section, a central section, and a distal section, wherein the central section is stiffer than the proximal section and the distal section.

6. The pump of claim 5, wherein the central section has a wall thickness that is greater than a wall thickness of the proximal section and the distal section.

7. The pump of claim 5, wherein the distal section is attached to the proximal section at the central section.

8. The pump of claim 5, wherein the proximal section includes a male shaped fitting configured to mate with a female shaped fitting of the distal section.

9. The pump of claim 5, further comprising an atraumatic tip coupled to the distal section.

10. The pump of claim 5, further comprising a guidewire lumen disposed within the distal tip.

11. The pump of claim 10, further comprising a self-sealing septum disposed at a distal end of the guidewire lumen.

12. The pump of claim 11, wherein the self-sealing septum is configured to be punctured to allow a guidewire to pass through.

13. The pump of claim 12, wherein the self-sealing septum is configured to re-seal itself when the guidewire is removed.

14. A method of delivering an intravascular blood pump, the method comprising:
piercing a self-sealing septum of a distal tip with a guidewire to insert the guidewire into the blood pump;
positioning the blood pump at a target tissue location with the guidewire; and
retracting the guidewire into the distal tip to cause the self-sealing septum to re-seal.
